SEQUENCE LISTING (3 sequences)

SEQ ID NO: 1 - protein, 349 aa, Artificial
  Sequenza C6-UG
DIVMSQSPSSLAVSVGEKVTMSCKSSQSLLYSSNQKNYLAWYQQRPGQSPKLLIYWASTGESGVPDRFTGSGSGTDFTLTISSVKAEDLAVYYCQQYYSYPLTFGAGTKLELKGSTSGSGKPGSGEGSSKGEVQLVESGGGLVQPKGSLKISCAASGLTFNTYAMNWVRQAPRKGLEWVARIRSKSNNYATYYADSVKDRFTISRDDSQSMLYLQMNNLKTEDTAMYYCVKQGGNSLYWYFDVWGAGTTVTVSSGSSSSGSSSSGSSSSGGSSSDICPGFLQVLEALLMESESGYVASLKPFNPGSDLQNAGTQLKRLVDTLPQETRINIMKLTEKILTSPLCKQDLRF

SEQ ID NO: 2 - protein, 335 aa, Artificial
  Sequenza D2E7-UG
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGDGSSGGSGGASDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKEFSSSSGSSSSGSSSSGGSSSDICPGFLQVLEALLMESESGYVASLKPFNPGSDLQNAGTQLKRLVDTLPQETRINIMKLTEKILTSPLCKQDLR

SEQ ID NO: 3 - protein, 607 aa, Artificial
  Sequenza C6-UG-D2E7
DIVMSQSPSSLAVSVGEKVTMSCKSSQSLLYSSNQKNYLAWYQQRPGQSPKLLIYWASTGESGVPDRFTGSGSGTDFTLTISSVKAEDLAVYYCQQYYSYPLTFGAGTKLELKGSTSGSGKPGSGEGSSKGEVQLVESGGGLVQPKGSLKISCAASGLTFNTYAMNWVRQAPRKGLEWVARIRSKSNNYATYYADSVKDRFTISRDDSQSMLYLQMNNLKTEDTAMYYCVKQGGNSLYWYFDVWGAGTTVTVSEFSSSSGSSSSGSSSSGGSSSDICPGFLQVLEALLMESESGYVASLKPFNPGSDLQNAGTQLKRLVDTLPQETRINIMKLTEKILTSPLCKQDLRFAAASSSSGSSSSGSSSSGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGDGSSGGSGGASDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

(19) United States
(12) Patent Application Publication
(10) Pub. No.: US 2013/0189735 A1
(43) Pub. Date: Jul. 25, 2013

(54) Process for engineering polyvalent, polyspecific fusion proteins using uteroglobin as skeleton and so obtained products.

(71) Applicant: Luciano Zardi, Camogli (IT)

(72) Inventor: Luciano Zardi, Camogli (IT)

(21) Appl. No.: 13/145,245

(22) PCT Filed: Jan. 18, 2010
(86) PCT No.: PCT/EP10/50501

§ 371 Date: Sep. 19, 2011

(30) Foreign Application Priority Data
Jan. 19, 2009 (IT) FI2009A000006

(51) Int. Cl. C07K 19/00

(52) U.S. Cl. CPC C07K 19/00
USPC 435/69.6; 435/69.7; 530/350; 435/375

(57) ABSTRACT

It is described a processes for generating stable and soluble polyvalent and polyspecific fusion proteins based on the use of uteroglobin (UG) as a reaction skeleton; proteins as above defined produced with said process are also described.

### FIELD OF THE INVENTION

The present invention refers to the field of processes for generating stable and soluble polyvalent and poly-specific fusion proteins. In particular we report here a novel procedure based on the use of uteroglobin.

### STATE OF THE ART

The generation of recombinant poly-valent and/or poly-specific fusion proteins as components in novel drugs is still hindered by factors that limit their production, storage and use, chief of which are the resulting proteins' instability or inadequate solubility. Here we describe a novel approach based on the use of uteroglobin (UG) as a skeleton for the generation of soluble and stable recombinant fusion protein proteins.

Human UG is a small (15.8 KDa), globular, non-glycosylated, homodimeric secreted protein, which was discovered independently by two groups in the 1960s in rabbit uterus (Krishnan, R. S. & Daniel, J.C. Jr. “Blastokinin”: inducer and regulator of blastocyst development in the rabbit uterus.” Science. 1967. 158, 490-492. Beier, H. M. “Uteroglobin: a hormone-sensitive endometrial protein involved in blastocyst development.” Biochim Biophys Acta. 1968. 160, 289-291) and it is the first member of a new superfamily of proteins, the so-called Secretoglobins (Scgb) (Klug, J. et al. The Uteroglobin/Clara cell protein family: Nomenclature Committee Report. In Mukherjee AB and Chilton BS eds. The Uteroglobin/Clara Cell Protein Family. Ann NY Acad Sci 2000; 923: 348-354). The mucosal epithelium of virtually all organs that communicate with the external environment express UG; it is present in the blood at a concentration of about 15 microgram per ml, and is found in urine and in other body fluids. The UG monomer is composed of about 70 amino acids, depending on the species, and is organized in a four-alpha helices secondary structure; the two subunits are joined in an anti-parallel fashion by disulfide bridges established between two highly conserved cysteine residues in amino and carboxi-terminal positions (Morize, I. et al. Refinement of the C222(1) crystal form of oxidized uteroglobin at 1.34 A resolution. J Mol Biol. 1987. 194, 725-739.) (see FIG. 1). The exact functions of UG are not yet clear, but the protein has been reported to have anti-inflammatory properties due to its ability to inhibit the soluble phospholipase A2 (Mukherjee, A. B., Zhang, Z. & Chilton, B. S. Uteroglobin: a steroid-inducible immunomodulatory protein that founded the Secretoglogin superfamily. Endocr Rev. 2007. 28, 707-725).

UG's high solubility and stability to pH and temperature variations, its resistance to proteases and its homodimeric structure prompted us to consider the protein as a candidate skeleton for the generation of polyvalent and polyspecific recombinant proteins with good properties of stability and solubility.

We demonstrate here that the use of UG provide a general method for the generation of covalently linked bivalent and tetravalent antibodies, either monospecific or bispecific, as well as of different kinds of fusion proteins with generally enhanced properties of solubility and stability compared to identical fusion proteins in which UG is not used.

### SUMMARY OF THE INVENTION

We describe here the use of UG as a skeleton for the production recombinant fusion proteins, bivalent, tetravalent and tetravalent dual-specific.

As Examples we describe here the use of UG in the production of:

1. a bivalent antibody using the variable fragments as single chain (scFv) of the monoclonal murin antibody C6 (see Italian Patent Application FI2008A000240) specific to the isoform of fibronectine (FN) associated to angiogenensis and containing the extradomain B (EDB) B-FN.

2. a bivalent antibody using the scfv D2E7, a human antibody able to neutralize the cytotoxic activity of TNF-alpha (Tracey, D., Klareskog, L., Sasso, E. H., Salfeld, J. G. & Tak, P. P. Tumor necrosis factor antagonist mechanisms of action: a comprehensive review. Pharmacol Ther. 2008. 117, 244-279)

3. a teravalent dual specific antibody composed of C6 and D2E7.

Of these molecules we describe here the production starting from the various cDNA fragments, the characterization, properties, and the biological activity. These results demonstrate as the use of appropriate protein sequences in the construction of recombinant fusion protein may modify the solubility and stability.

### BRIEF DESCRIPTION OF THE DRAWINGS

FIG. 1

(A-D): represents schematically the molecule of UG (A) and of the three fusion proteins described in the examples produced using UG as skeleton (B-D).

FIG. 2

(A) Scheme of the cDNA construct of C6-UG; (B-C)characterization of the purified C6-UG: (B)SDS-PAGE analysis of the purified C6-UG before and after lyophilization and (C) Size exclusion chromatography profile (Superdex200); (D) Biodistribution experiments of the radioiodinated C6-UG in human melanoma SK-MEL 28 tumor-bearing nude mice. The studies were performed when the tumours were about 0.5 centimeter cube. The figure shows the percentage of the injected dose per gram of tissue (% ID/g) both in the tumour and in the blood and the ratio between the % ID/g of the tumour and blood.

FIG. 3.

(A) Scheme of the cDNA construct of D2E7-UG; (B) SDS-PAGE analysis of the purified fusion protein D2E7-UG; (C) The size exclusion chromatography profile (Superdex200) of purified D2E7-UG.

FIG. 4.

(A) Scheme of the cDNA construct of C6-UG-1; (B) SDS-PAGE analysis of the purified fusion protein C6-UG-D2E7; (C) Size exclusion chromatography profile (Superdex200) of purified C6-UG-D2E7; (D) Immunoreactivity of the two antibody moieties of the C6-UG-D2E7 molecule with the respective antigens, TNF-alpha and the FN recombinant fragment containing both the type III repeats EDB and 8; (E) Neutralization of the hTNF-alpha cytotoxicity, on L-M mouse fibroblasts, by C6-UG-D2E7.

FIG. 5

The reaction of C6-UG-D2E7 with TNF-alpha in solid phase did not reduce the immunoreactivity of C6 (A-B); (C) C6-UG-D2E7 neutralizes TNF-alpha also when it is bound to the FN antigen (in situ neutralization). On the contraty D2E7-UG does not inhibit TNF-alpha since it is not able to bind to the FN epitope and consequently it is completely removed by the washing.

### DETAILED DESCRIPTION OF THE INVENTION

The present invention makes available a new process for the production of polyvalent, and/or polyspecific proteins using UG as central skeleton. In fact the use of UG as a linker provides a general method for the generation of bivalent and tetravalent dual-specific antibodies, as well as of different kinds of fusion proteins. Moreover the introduction of the UG molecule, normally enhances the stability and solubility of the fusion proteins.

It was in fact established that by ligating the DNA sequences coding for biologically active molecules to one or to both ends of the DNA coding for UG, constructs for the expression of covalently bound bivalent and tetravalent dual-specific fusion proteins can be generated and efficiently produced in mammalian cells.

The majority of fusion proteins generated using UG shows a solubility that allows their lyophilization and reconstitution without any aggregation or loss in protein or biological activity.

Following the above said process according to the invention dimeric and tetrameric molecules were engineered and characterized, both in vitro and in vivo, as reported in the following examples; these molecules are obviously only a few examples of the manifold possibilities offered by this approach.

According to the invention for “biologically active molecules” as above defined it is intended for example: antibodies, fragments of antibodies, cytokines, chemokines, molecules with antiinflammatory activity, molecules with cytotoxic activity, molecules able to induce regeneration of tissues and, molecules with immunosuppressive activity etc.

The process of the invention comprises the following steps:

a) Generation of the cDNA constructs using as the central core the cDNA of UG and ligating cDNAs coding for different biologically active molecules.

b) Transfection of mammalian cells using the above cDNAs and selection of the producing clones.

c) Purification of the fusion proteins from the spent media of the transfected cells.

d) Characterization of the purified fusion proteins.

The invention will now be better illustrated in the light of the following examples.

### Materials and Methods

cDNA Constructs, Expression and Purification of Fusion Proteins.

Uteroglobin cDNA sequence, provided by GenScript Corporation (Piscataway, N.J.), was inserted into the vector pProEX-1. All PCRs reactions were realized with high fidelity PWO DNA Polymerase (Roche) according to the manufacturer's instructions. All restriction enzymes were from Roche Diagnostic. All the PCR products and the digested cDNA fragments were purified with the High Pure PCR Purification Kit (Roche Diagnostic). The digested DNA fragments were purified by gel agarose and gel extraction with the Qiaquick Gel Extraction Kit (Qiagen, Hilden, Germany). Clones were screened by PCR. The plasmid DNAs were purified from positive clones using the PureLink HiPure Plasmid Filter Maxiprep kit (Invitrogen) and the DNA sequences were confirmed by the DNA sequencing of both strands. The purified construct were used to transfect CHO K1 cells (American Tissue Type Culture Collection, ATCC, Rockville, Md.) using Lipofectamine 2000 CD Reagent (Invitrogen) according to the manufacture. Transfectomas were grown in RPMI 1640 (Euroclone) supplemented with 10% FBS (Biochrom AG; Berlin, Germany) and 4 mM L-glutamine (Invitrogen) and selected using 500 μg/ml of Geneticin (G418, Calbiochem, San Diego, Calif.).

The supernatants of the G418 resistant clones were screened for the production of the fusions proteins by using the enzyme linked immunosorbent assay (ELISA). The recombinant peptide composed of the type III homology repeats 7-EDB-8-9 (Carnemolla, B. et al. Phage antibodies with pan-species recognition of the oncofoetal angiogenesis marker fibronectin ED-B domain. Int J Cancer. 1996. 68, 397-405.) was used as antigen for fusion proteins containing C6 antibody and recombinant humanTNFalpha (Peprotech, Rocky Hill, N.J.) for fusion proteins containing D2E7.

A rabbit polyclonal anti-mouse UG IgG was used as secondary antibody and a peroxidase-conjugated anti-rabbit immunoglobulin G (IgG) polyclonal (Pierce, Rockford, Ill.) as tertiary antibody.

Fusion proteins were immunopurified from the conditioned media of the cells on 7-EDB-8-9 (Carnemolla, B. et al. Phage antibodies with pan-species recognition of the oncofoetal angiogenesis marker fibronectin ED-B domain. Int J Cancer. 1996. 68, 397-405.) or recombinant hTNFalpha (Peprotech) conjugated to Sepharose 4B (Amersham Pharmacia Biotech, Uppsala, Sweden). Immunopurified proteins were analyzed in native conditions by fast protein liquid chromatography on a Superdex200 column (Amersham Pharmacia Biotech) and by sodium dodecyl sulfate-polyacrylamide gel electrophoresis (SDS-PAGE; 4%-12% gradient) under reducing and non reducing conditions.

### D2E7-UG

The cDNA sequence encoding for D2E7 (Safield et al. 2003, U.S. Pat. No. 6,090,382), linker and UG, cloned into pcDNA3.1, was provided by Genscript Corporation.

### C6-UG

For the generation of the cDNA encoding for C6-UG, we amplified UG sequence preceded by a sequence encoding for a 15 aminoacid-linker (Borsi, L. et al. Selective targeted delivery of TNFα to tumor blood vessels. Blood. 2003. 102, 4384-4392). by PCR from the mouse UG cDNA. This cDNA fragment was digested using BspEI and EcoRI and inserted in the pcDNA3.1 clone containing the C6 scFv previously digested using the same restriction enzymes.

### C6-UG-D2E7

For the generation of C6-UG-D2E7 we amplified by PCR the sequence encoding for the signal peptide, C6, linker and uteroglobin minus the stop codon from the construct pcDNA3.1/C6-UG above described. The obtained sequence was digested HindIII/NotI. To obtain the D2E7 sequence preceded by the linker we amplified by PCR the cDNA of D2E7 with a primer containing the complete sequence of the linker. The obtained DNA was digested NotI/XbaI. The two digested DNA fragments, C6-UG-linker and linker-D2E7, were ligated together with HindIII/XbaI digested pcDNA3.1 to form pcDNA3.1/C6-UG-D2E7. All the above obtained cDNA constructs were used to transform DH5□ competent bacteria cells and clones were selected in Luria Bertani medium (LB) with 100 □g/ml of ampicillin.

### Radioiodination and Biodistribution Experiments of C6-UG.

C6-UG was radioiodinated as previously (Borsi, L. et al. Selective targeting of tumoral vasculature: comparison of different formats of an antibody (L19) to the ED-B domain of fibronectin. Int. J. of Cancer. 2002. 102, 75-85.) The purified fusion protein was radiolabeled with iodine 125 using the Iodogen method (Pierce, Rockford, Ill.). The immunoreactivity of radiolabeled fusion protein was more than 90%. Nude mice with subcutaneously implanted SKMeI28 were injected intravenously with about 10 μg (4 μCi; 0.148 MBq) protein in 100 μL saline solution. Three animals were used for each time point. Mice were killed at 4, 24, 48 and 96 hours after injection. The organs were weighed and the radioactivity was counted. Targeting results of representative organs are expressed as percent of the injected dose per gram of tissue (% ID/g).

### TNFalpha Neutralizing Cytotoxic Activity of D2E7 Containing Fusion Proteins.

The ability of the D2E7 containing fusion proteins to neutralize hTNFalpha activity was assessed by using the cytotoxicity test on L-M fibroblasts (ATCC, Rockville, Md.) as previously described (Corti, A., Poiesi, C., Merli, S. & Cassani, G. “Tumor necrosis factor a quantification by ELISA and bioassay: effects of TNF receptor (p55) complex dissociation during assay incubations”. J Immunol Methods. 1994. 177, 191-198). The L_M cells were treated with recombinant TNF 1 pM reprotech, Rocky Hill, N.J.) in the presence of 0.01 to 1500 pM C6-UG-D2E7 or D2E7-UG.

### Example 1

### C6-UG

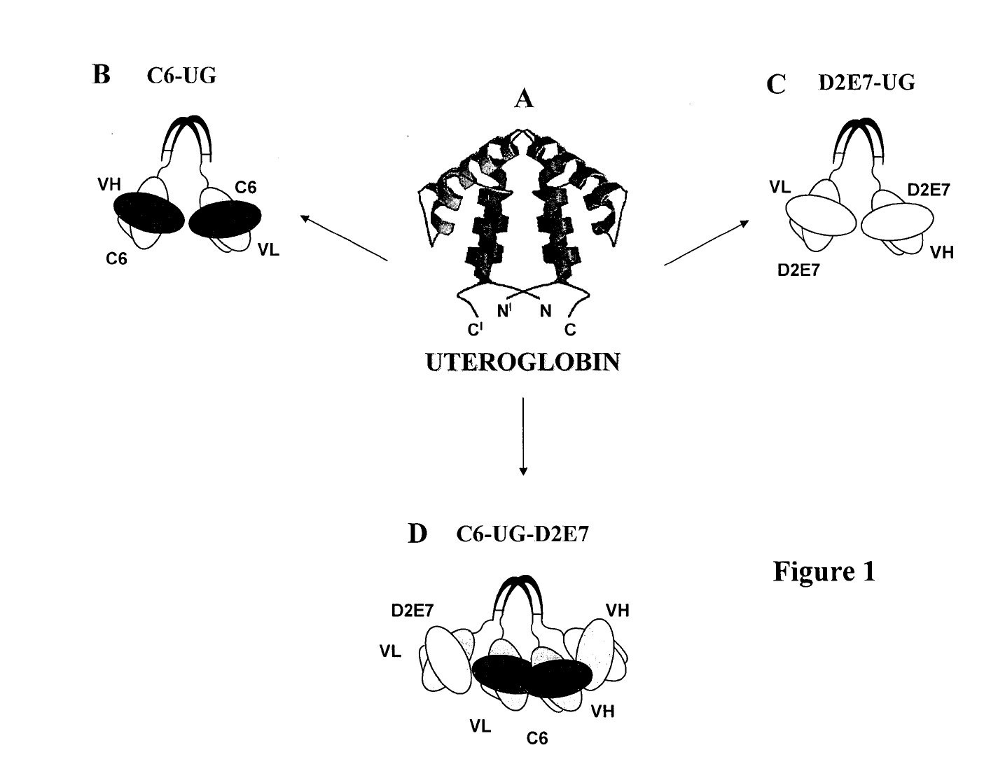

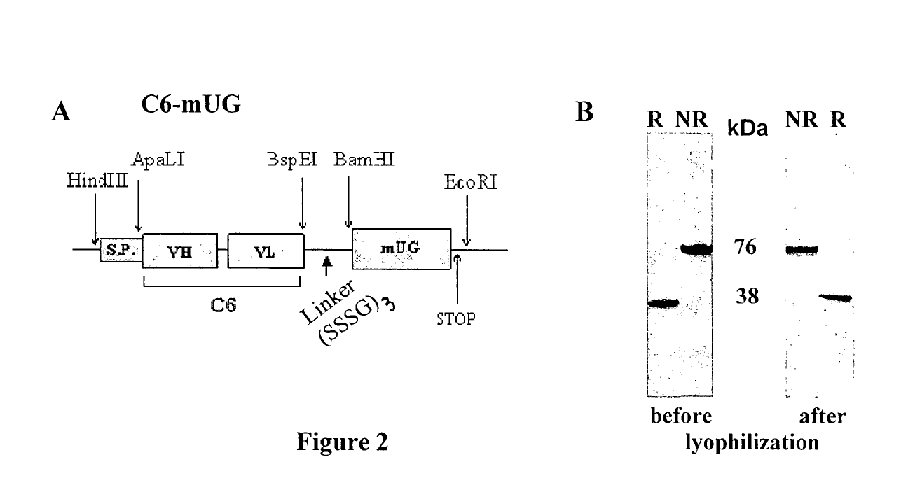

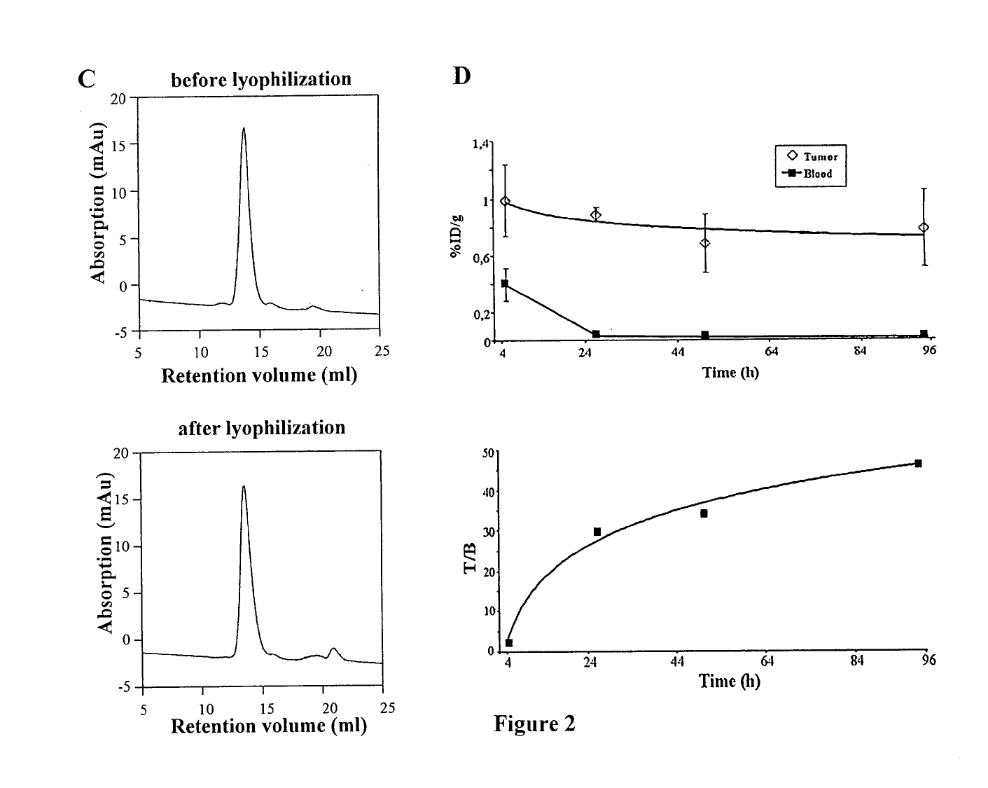

As is shown in FIGS. 1B and 2A, we prepared cDNA constructs between the scFv C6 and UG by ligating the cDNA of the murine scFv C6 (Balza et al. Submitted) in the 5′ of the UG cDNA in order to produce the divalent C6. FIG. 2 shows the structure of the cDNA construct (A) of C6-UG used to transfect CHO cells that growth in the animal protein-free media ProCHO5 (Lonza, Verviers, Belgium) and produce about 4 mg/liter of recombinant protein that can be efficiently purified by affinity chromatography either using the fibronectin fragment constituted by the type III repeats 7-EDB-8-9 (containing the antigen of C6) or protein A.

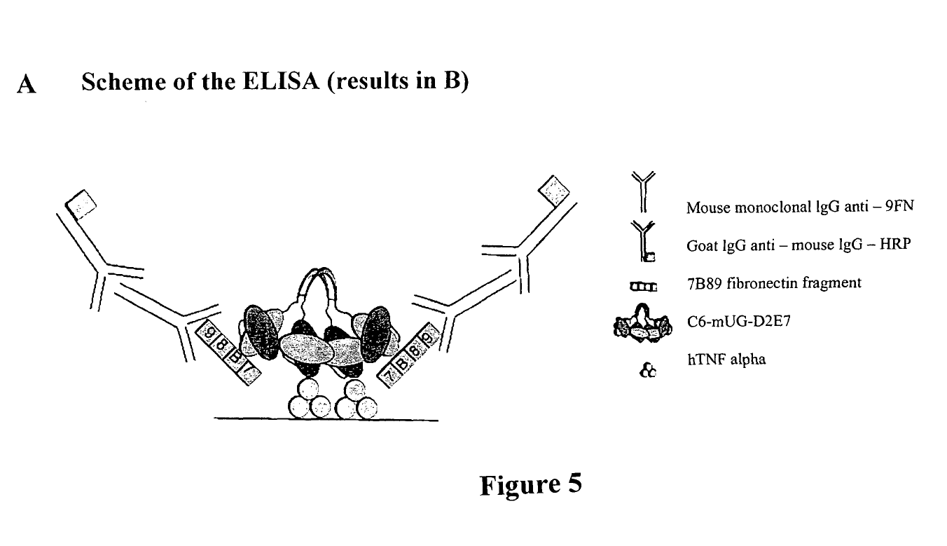

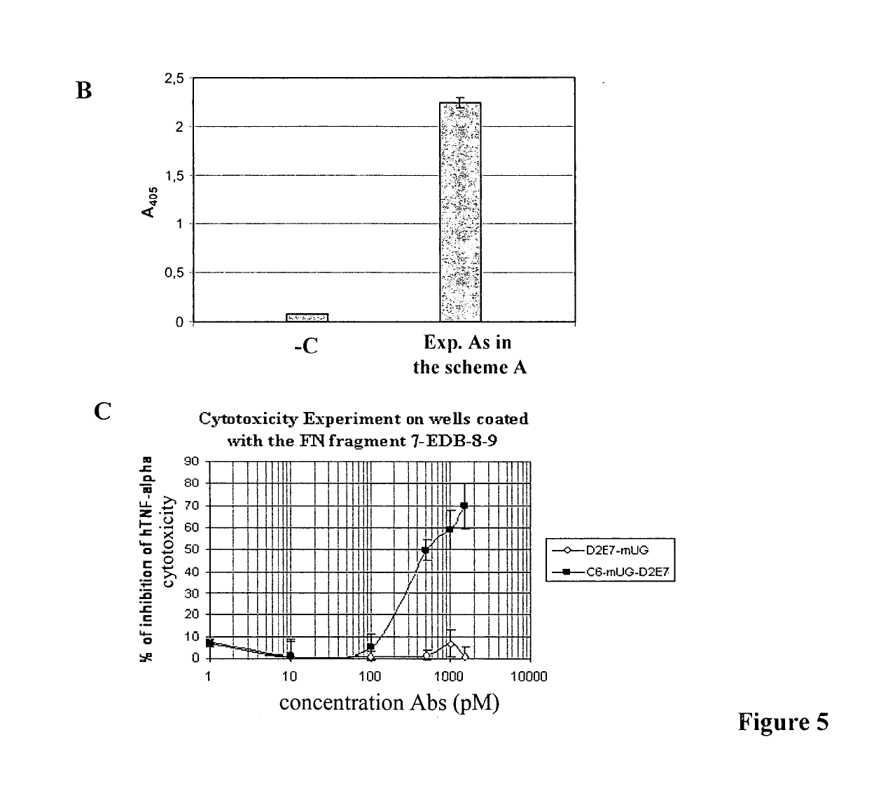

In SDS-PAGE the fusion protein migrates as homodimer in non reducing conditions and as monomer in reducing conditions showing the expected sizes of about 76 and 38 KDa, respectively. In non reducing conditions the molecule was more than 95 percent covalently linked dimer (FIG. 2B). The size exclusion chromatography (SEC) profile showed a single peak with a retention volume corresponding to the molecular mass of the homodimer (FIG. 2C). The proteins is very soluble, and it was possible to have solution in PBS at a concentration over 1 mg/ml and to lyophilize and reconstitute this protein without any loss or formation of aggregates (FIGS. 2B and 2C). Tumor targeting experiments, were carried out in tumor-bearing mice using radioiodinated C6-UG. FIG. 2D shows the percentage of the injected dose per gram of tissue (% ID/g) in the tumour and in blood. The results indicate a very fast clearance of C6-UG from blood. FIG. 5E shows the ratios of the % ID/g of tumor and of blood, 96 hours after injection of the radioiodinated protein this ratio was about 50. The ratio of the % ID/g in the tumor and other organs were in all cases higher than 10.

Sequence: C6-mUG:
(SEQ ID N^{o} 1)
DIVMSQSPSSLAVSVGEKVTMSCKSSQSLLYSSNQKNYLAWYQQRPGQS
PKLLIYWASTGESGVPDRFTGSGSGTDFTLTISSVKAEDLAVYYCQQYY
SYPLTFGAGTKLELKGSTSGSGKPGSGEGSSKGEVQLVESGGGLVQPKG
SLKISCAASGLTFNTYAMNWVRQAPRKGLEWVARIRSKSNNYATYYADS
VKDRFTISRDDSQSMLYLQMNNLKTEDTAMYYCVKQGGNSLYWYFDVWG
AGTTVTVS (C6)
SGSSSSGSSSSGSSSSGGS (linker)
SSDICPGFLQVLEALLMESESGYVASLKPFNPGSDLQNAGTQLKRLVDT
LPQETRINIMKLTEKILTSPLCKQDLRF (UG)

### Example 2

### D2E7-UG

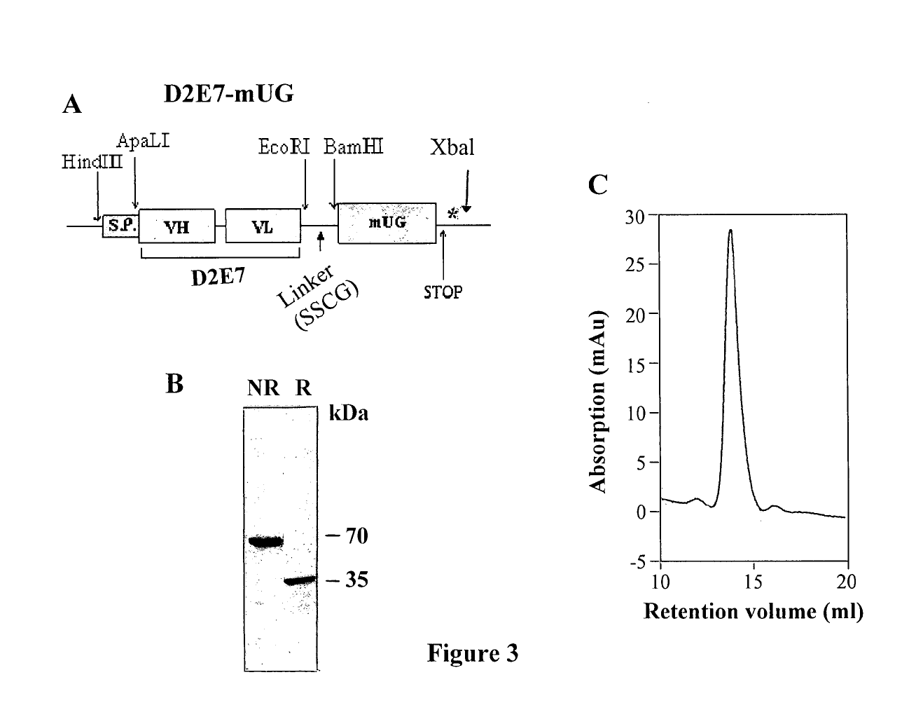

We prepared the cDNA construct encoding for D2E7-UG by ligating the cDNA of D2E7 scFv at the 5′ end of UG cDNA in order to obtain the divalent format of D2E7, as it is shown in FIGS. 1C and 3A. The cDNA construct was used to transfect CHO cells and the fusion protein was purified from the conditioned medium of transfected cells by immunoaffinity chromatography on hTNF-alpha conjugated to sepharose 4B. As is shown in FIG. 3B the purified fusion protein migrates as a homodimer in non-reducing condition with the expected apparent molecular mass of about 72 KDa and as monomer of 36 KDa, in reducing condition. The SEC profile, FIG. 3C, shows a single peak with a retention volume corresponding to the molecular mass of D2E7-UG dimer.

Sequence: D2E7-mUG
(SEQ ID N^{o} 2)
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSGDGSSGGSGGASDIQMTQSPSSLSAS
VGDRVTITCRASQGIRNYLAWYQQKPGKAPKLLIYAASTLQSGVPSRFS
GSGSGTDFTLTISSLQPEDVATYYCQRYNRAPYTFGQGTKVEIK
(DEE7)
EFSSSSGSSSSGSSSSGGS (linker)
SSDICPGFLQVLEALLMESESGYVASLKPFNPGSDLQNAGTQLKRLVDT
LPQETRINIMKLTEKILTSPLCKQDLRF (UG)

### Example 3

### C6-UG-D2E7

D2E7 is a human scFv able to inhibit TNF-alpha activity, and is marketed as a complete IgG under the brand name Humira for the treatment of rheumatoid arthritis (RA) and other autoimmune diseases (Tracey et al. 2008). Given that the oncofetal FN isoform containing EDB is over-expressed in RA tissues (Kriegsmann, J. et al. Expression of fibronectin splice variants and oncofetal glycosylated fibronectin in the synovial membranes of patients with rheumatoid arthritis and osteoarthritis. Rheumatol Int 2004. 24 25-33).we generated a dual-specific tetravalent molecule using as a skeleton the UG molecule of and the scFvs C6 (specific for B-FN) and D2E7 (inhibiting TNF-alpha). This molecule offers the possibility to selectively deliver D2E7 to the diseased tissues, thereby achieving an “in situ” inhibition of the TNF-alpha activity. Seeing that UG also is an anti-inflammatory molecule, this fusion protein theoretically constitutes a powerful “in situ” anti-inflammatory drug.

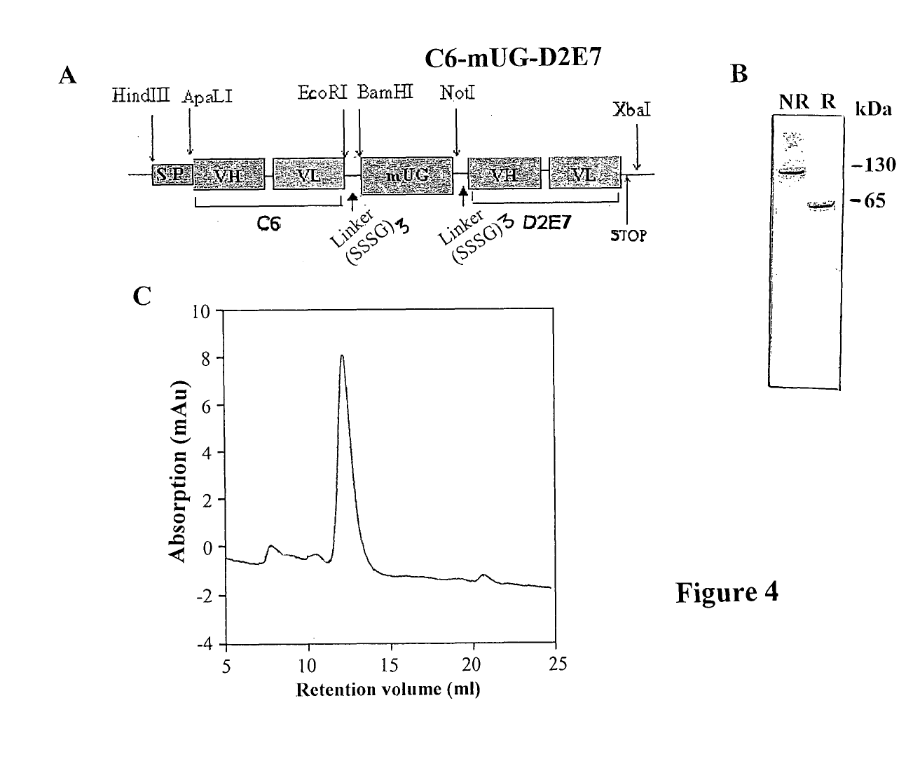

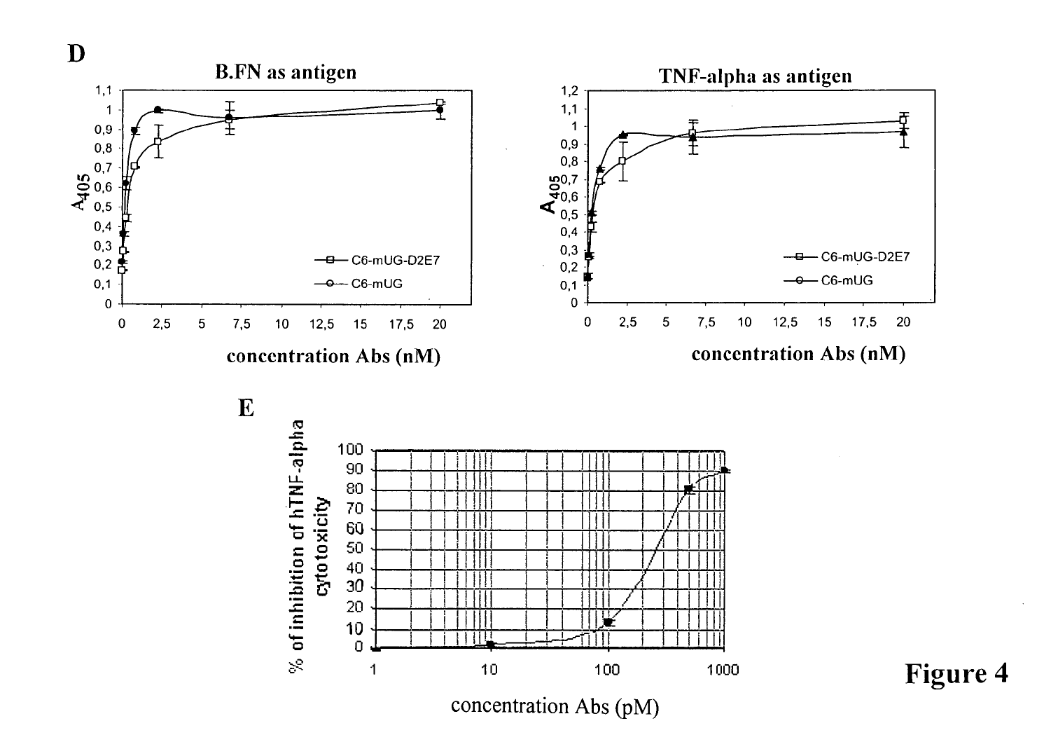

As is shown in FIG. 4A we prepared the cDNA construct of C6-UG-D2E7 by ligating the cDNA of the scFv C6 and the cDNA of the scFv D2E7 at the 5′ and 3′ ends, respectively, of UG cDNA. FIGS. 4B-E show the characterization of the purified dual-specific tetravalent molecule C6-UG-D2E7. In SDS-PAGE (FIG. 4B) the protein migrated as a homodimer in non reducing conditions, showing the expected size of about 130 KDa, and as a monomer with a size of 65 KDa in reducing conditions. The SEC profile (FIG. 4C) showed a main peak with a retention volume corresponding to the apparent molecular mass of about 130 KDa. The immunoreactivity properties of C6-UG-D2E7 were tested by ELISA against the two antigens, 7-EDB-8-9 and TNF-alpha. FIG. 4D shows that C6-UG and C6-UG-D2E7 reacted equally well with 7-EDB-8-9, and that D2E7-UG and C6-UG-D2E7 reacted equally well with TNF-alpha, thereby demonstrating that the two scFvs within the C6-UG-D2E7 molecule do not interfere with each other. FIG. 4E depicts the ability of inhibiting TNF-alpha cytotoxicity of the dual specific tetravalent C6-UG-D2E7.

We also demonstrated by ELISA that each binding domain could function independently without interfering with each other even when a scFv is bound at its antigen in solid phase (5A and 5B). We coated ELISA wells with TNF-alpha: incubated with C6-UG-D2E7 that binds to the antigen using its D2E7 antibody. The excess of antibody was washed away and the FN fragment composed of the type III repeat 7-EDB-8-9 was added to the well. This fragment binds the C6 moieties and was then detected using a monoclonal antibody specific for the FN type III repeat 9. The results demonstrated that even when a scFv is occupied by the antigen in solid phase, the other is still free to react with the antigen. FIG. 5A shows the scheme of the tested used and FIG. 5B shows the results.

These results show that also when one of the two scFv is bound to the antigen in solid phase, the second scFv is tisII free to react with its antigene.

These results were confirmed by cytotoxicity experiments on L-M fibroblasts (FIG. 5C) demonstrating that also when C6-UG-D2E7 is bound, by the scFV C6, to the FN isoform containing EDB, is able to inhibit the cytotoxic activity of TNF-alpha. In fact to mimic the targeted delivery of D2E7 on BNF containing tissues, C6-UG-D2E7 and D2E7-UG inhibitory activity of the TNFalpha cytotoxicity was evaluated on L-M cells plated on 7-EDB-8-9 pre-coated cell culture plates: after cells incubation with the two fusion proteins (D2E7-UG and C6-UG-D2E7) and washing out of the excess, hTNFapha was added (FIG. 5D). The obtained result demonstrates that even when C6 is bound to its antigen the anti-TNFalpha moieties D2E7 neutralize hTNF-alpha. Being not able to bind to the FN substrate, the D2E7-UG was completely washed out and no TNF-alpha inhibition was observed. We used D2E7-Ug as negative control.

Sequence: C6-mUG-D2E7
(SEQ. ID. N^{o} 3)
DIVMSQSPSSLAVSVGEKVTMSCKSSQSLLYSSNQKNYLAWYQQRPGQS
PKLLIYWASTGESGVPDRFTGSGSGTDFTLTISSVKAEDLAVYYCQQYY
SYPLTFGAGTKLELKGSTSGSGKPGSGEGSSKGEVQLVESGGGLVQPKG
SLKISCAASGLTFNTYAMNWVRQAPRKGLEWVARIRSKSNNYATYYADS
VKDRFTISRDDSQSMLYLQMNNLKTEDTAMYYCVKQGGNSLYWYFDVWG
AGTTVTVS (C6)
EFSSSSGSSSSGSSSSGGS (linker)
SSDICPGFLQVLEALLMESESGYVASLKPFNPGSDLQNAGTQLKRLVDT
LPQETRINIMKLTEKILTSPLCKQDLRF (UG)
AAASSSSGSSSSGSSSSG (linker)
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSGDGSSGGSGGASDIQMTQSPSSLSAS
VGDRVTITCRASQGIRNYLAWYQQKPGKAPKLLIYAASTLQSGVPSRFS
GSGSGTDFTLTISSLQPEDVATYYCQRYNRAPYTFGQGTKVEIK
(D2E7)

1-7. (canceled)

8. A process for manufacture of a polyvalent, poly-specific fusion protein, comprising ligating a cDNA molecule which encodes uteroglobin to a cDNA molecule which encodes a protein, transforming or transfecting a cell with the resulting cDNA molecule, and culturing said cell under conditions favoring production of said polyvalent, polyspecific fusion protein expressed by said cDNA molecule.

9. The process of claim 8, comprising ligating a cDNA molecule which encodes a protein to each end of the cDNA molecule which encodes uteroglobin.

10. The process of claim 8, wherein said cell is a mammalian cell.

11. The process of claim 8, wherein said protein is selected from the group consisting of an antibody, a binding fragment of an antibody, a cytokine, a chemokine, a protein with anti-inflammatory activity, a protein with cytotoxic activity, and a protein with immunosuppressive activity.

12. The process of claim 10, further comprising purifying said fusion protein from medium in which said cell is cultured.

13. The process of claim 12, further comprising lyophilizing said fusion protein.

14. The process of claim 8, wherein said fusion protein comprises from 2-4 antibody molecules, each of which binds to a different target molecule.

15. A fusion protein consisting of the amino acid sequence of SEQ ID NO: 1, SEQ ID NO: 2, or SEQ ID NO: 3.

16. A method for inhibition of TNF-α with a fusion protein produced via the method of claim 8, wherein said fusion protein comprises an antibody which binds specifically to the extracellular matrix of a tissue in which TNF-α is expressed, an anti-inflammatory protein, an immunosuppressive protein, and a protein which inhibits a pro-inflammatory cytokine.

17. The method of claim 16, wherein said fusion protein consists of the amino acid sequence set forth in SEQ ID NO: 3.
